(19) United States
(12) Patent Application Publication
(10) Pub. No.: US 2017/0105856 A1
(43) Pub. Date: Apr. 20, 2017

(54) NON-CIRCULAR RADIOPAQUE MARKERS AND METHODS FOR ATTACHING A MARKER TO A SCAFFOLD

(71) Applicant: Abbott Cardiovascular Systems Inc., Santa Clara, CA (US)

(72) Inventors: Ryan Vaughan, Murrieta, CA (US); Matthew J. Chludzinski, Poway, CA (US); John A. Simpson, Carlsbad, CA (US)

(21) Appl. No.: 14/885,926

(22) Filed: Oct. 16, 2015

(51) Int. Cl. A61F 2/90

(52) U.S. Cl. CPC A61F 2/90; A61F 2250/0098; A61F 2220/0033; A61F 2240/001

(57) ABSTRACT

A scaffold includes a marker connected to a strut. The marker is retained within the strut by a tongue-and-groove connection. The marker is attached to the strut by a process that includes pressing a non-circular marker into a rectangular hole of the scaffold strut. The strut sidewalls are restrained to produce the tongue and groove connection.

### BACKGROUND OF THE INVENTION

Field of the Invention

The present invention relates to bioresorbable scaffolds; more particularly, this invention relates to bioresorbable scaffolds for treating an anatomical lumen of the body.

Description of the State of the Art

Radially expandable endoprostheses are artificial devices adapted to be implanted in an anatomical lumen. An “anatomical lumen” refers to a cavity, or duct, of a tubular organ such as a blood vessel, urinary tract, and bile duct. Stents are examples of endoprostheses that are generally cylindrical in shape and function to hold open and sometimes expand a segment of an anatomical lumen. Stents are often used in the treatment of atherosclerotic stenosis in blood vessels. “Stenosis” refers to a narrowing or constriction of the diameter of a bodily passage or orifice. In such treatments, stents reinforce the walls of the blood vessel and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stent. “Delivery” refers to introducing and transporting the stent through an anatomical lumen to a desired treatment site, such as a lesion. “Deployment” corresponds to expansion of the stent within the lumen at the treatment region. Delivery and deployment of a stent are accomplished by positioning the stent about one end of a catheter, inserting the end of the catheter through the skin into the anatomical lumen, advancing the catheter in the anatomical lumen to a desired treatment location, expanding the stent at the treatment location, and removing the catheter from the lumen.

The following terminology is used. When reference is made to a “stent”, this term will refer to a permanent structure, usually comprised of a metal or metal alloy, generally speaking, while a scaffold will refer to a structure comprising a bioresorbable polymer, or other resorbable material such as an erodible metal, and capable of radially supporting a vessel for a limited period of time, e.g., 3, 6 or 12 months following implantation. It is understood, however, that the art sometimes uses the term “stent” when referring to either type of structure.

Scaffolds and stents traditionally fall into two general categories—balloon expanded and self-expanding. The later type expands (at least partially) to a deployed or expanded state within a vessel when a radial restraint is removed, while the former relies on an externally-applied force to configure it from a crimped or stowed state to the deployed or expanded state.

Self-expanding stents are designed to expand significantly when a radial restraint is removed such that a balloon is often not needed to deploy the stent. Self-expanding stents do not undergo, or undergo relatively no plastic or inelastic deformation when stowed in a sheath or expanded within a lumen (with or without an assisting balloon). Balloon expanded stents or scaffolds, by contrast, undergo a significant plastic or inelastic deformation when both crimped and later deployed by a balloon.

In the case of a balloon expandable stent, the stent is mounted about a balloon portion of a balloon catheter. The stent is compressed or crimped onto the balloon. Crimping may be achieved by use of an iris-type or other form of crimper, such as the crimping machine disclosed and illustrated in US 2012/0042501. A significant amount of plastic or inelastic deformation occurs both when the balloon expandable stent or scaffold is crimped and later deployed by a balloon. At the treatment site within the lumen, the stent is expanded by inflating the balloon.

The stent must be able to satisfy a number of basic, functional requirements. The stent (or scaffold) must be capable of sustaining radial compressive forces as it supports walls of a vessel. Therefore, a stent must possess adequate radial strength. After deployment, the stent must adequately maintain its size and shape throughout its service life despite the various forces that may come to bear on it. In particular, the stent must adequately maintain a vessel at a prescribed diameter for a desired treatment time despite these forces. The treatment time may correspond to the time required for the vessel walls to remodel, after which the stent is no longer needed.

Examples of bioresorbable polymer scaffolds include those described in U.S. Pat. No. 8,002,817 to Limon, No. 8,303,644 to Lord, and No. 8,388,673 to Yang. FIG. 1 shows a distal region of a bioresorbable polymer scaffold designed for delivery through anatomical lumen using a catheter and plastically expanded using a balloon. The scaffold has a cylindrical shape having a central axis 2 and includes a pattern of interconnecting structural elements, which will be called bar arms or struts 4. Axis 2 extends through the center of the cylindrical shape formed by the struts 4. The stresses involved during compression and deployment are generally distributed throughout the struts 4 but are focused at the bending elements, crowns or strut junctions. Struts 4 include a series of ring struts 6 that are connected to each other at crowns 8. Ring struts 6 and crowns 8 form sinusoidal rings 5. Rings 5 are arranged longitudinally and centered on an axis 2. Struts 4 also include link struts 9 that connect rings 5 to each other. Rings 5 and link struts 9 collectively form a tubular scaffold 10 having axis 2 represent a bore or longitudinal axis of the scaffold 10. Ring 5d is located at a distal end of the scaffold. Crown 8 form smaller angles when the scaffold 10 is crimped to a balloon and larger angles when plastically expanded by the balloon. After deployment, the scaffold is subjected to static and cyclic compressive loads from surrounding tissue. Rings 5 are configured to maintain the scaffold's radially expanded state after deployment.

Scaffolds may be made from a biodegradable, bioabsorbable, bioresorbable, or bioerodable polymer. The terms biodegradable, bioabsorbable, bioresorbable, biosoluble or bioerodable refer to the property of a material or stent to degrade, absorb, resorb, or erode away from an implant site. Scaffolds may also be constructed of bioerodible metals and alloys. The scaffold, as opposed to a durable metal stent, is intended to remain in the body for only a limited period of time. In many treatment applications, the presence of a stent in a body may be necessary for a limited period of time until its intended function of, for example, maintaining vascular patency and/or drug delivery is accomplished. Moreover, it has been shown that biodegradable scaffolds allow for improved healing of the anatomical lumen as compared to metal stents, which may lead to a reduced incidence of late stage thrombosis. In these cases, there is a desire to treat a vessel using a polymer scaffold, in particular a bioabsorable or bioresorbable polymer scaffold, as opposed to a metal stent, so that the prosthesis's presence in the vessel is temporary.

Polymeric materials considered for use as a polymeric scaffold, e.g. poly(L-lactide) (“PLLA”), poly(D,L-lactide-co-glycolide) (“PLGA”), poly(D-lactide-co-glycolide) or poly(L-lactide-co-D-lactide) (“PLLA-co-PDLA”) with less than 10% D-lactide, poly(L-lactide-co-caprolactone), poly(caprolactone), PLLD/PDLA stereo complex, and blends of the aforementioned polymers may be described, through comparison with a metallic material used to form a stent, in some of the following ways. Polymeric materials typically possess a lower strength to volume ratio compared to metals, which means more material is needed to provide an equivalent mechanical property. Therefore, struts must be made thicker and wider to have the required strength for a stent to support lumen walls at a desired radius. The scaffold made from such polymers also tends to be brittle or have limited fracture toughness. The anisotropic and rate-dependent inelastic properties (i.e., strength/stiffness of the material varies depending upon the rate at which the material is deformed, in addition to the temperature, degree of hydration, thermal history) inherent in the material, only compound this complexity in working with a polymer, particularly, bioresorbable polymers such as PLLA or PLGA.

One additional challenge with using a bioresorbable polymer (and polymers generally composed of carbon, hydrogen, oxygen, and nitrogen) for a scaffold structure is that the material is radiolucent with no radiopacity. Bioresorbable polymers tend to have x-ray absorption similar to body tissue. A known way to address the problem is to attach radiopaque markers to structural elements of the scaffold, such as a strut, bar arm or link. For example, FIG. 1 shows a link element 9d connecting a distal end ring 5d to an adjacent ring 5. The link element 9d has a pair of holes. Each of the holes holds a radiopaque marker 11. There are challenges to the use of the markers 11 with the scaffold 10.

There needs to be a reliable way of attaching the markers 11 to the link element 9d so that the markers 11 will not separate from the scaffold during a processing step like crimping the scaffold to a balloon or when the scaffold is balloon-expanded from the crimped state. These two events—crimping and balloon expansion—are particularly problematic for marker adherence to the scaffold because both events induce significant plastic deformation in the scaffold body. If this deformation causes significant out of plane or irregular deformation of struts supporting, or near to markers the marker can dislodge (e.g., if the strut holding the marker is twisted or bent during crimping the marker can fall out of its hole). A scaffold with radiopaque markers and methods for attaching the marker to a scaffold body is discussed in US20070156230.

There is a continuing need to improve upon the reliability of radiopaque marker securement to a scaffold; and there is also a need to improve upon methods of attaching radiopaque markers to meet demands for scaffold patterns or structure that render prior methods of marker attachment in adequate or unreliable.

### SUMMARY OF THE INVENTION

What is disclosed are scaffolds having non-circular radiopaque markers and methods for attaching non-circular radiopaque markers to a strut, link or bar arm of a polymeric scaffold.

According to one aspect markers are re-shaped to facilitate a better retention within a marker hole. Examples include a marker shaped as a rectangle and having at least one pair of concave or convex surfaces, or an X-shaped marker having four flanges extending radially outward from a center. Each of these marker shapes may be made from a variety of well-known processes, such as by drawing a radiopaque wire through a die.

According to another aspect a marker is held within a hole of the link, or bar arm by a tongue and groove connection. The tongue and groove connection is formed in a preferred embodiment by applying a lateral constraint to the scaffold element when the marker is being inserted into the element. The hole for the marker has a rectangular opening, as opposed to a circular opening as in FIGS. 1 and 2.

According to another embodiment a rectangular radiopaque marker is inserted into a hole of a link, strut or bar arm without lateral restraining. This embodiment may also produce a tongue and groove engagement by virtue of resistance to bulging of side walls by; e.g., the link element made substantially thicker and/or wider surrounding the hole to increase its flexural stiffness near the hole.

According to another aspect a marker is inserted into the hole of a scaffold element, such as a link or strut, by a cold forged or swaging process.

According to another aspect of the invention a scaffold structure for holding a marker and method for making the same addresses a need to maintain a low profile for struts exposed in the bloodstream, while ensuring the marker will be securely held in the strut. Low profiles for struts mean thinner struts or thinner portions of struts. The desire for low profiles addresses the degree thrombogenicity of the scaffold, which can be influenced by a strut thickness overall and/or protrusion from a strut surface. Blood compatibility, also known as hemocompatibility or thromboresistance, is a desired property for scaffolds and stents. The adverse event of scaffold thrombosis, while a very low frequency event, carries with it a high incidence of morbidity and mortality. To mitigate the risk of thrombosis, dual anti-platelet therapy is administered with all coronary scaffold and stent implantation. This is to reduce thrombus formation due to the procedure, vessel injury, and the implant itself. Scaffolds and stents are foreign bodies and they all have some degree of thrombogenicity. The thrombogenicity of a scaffold refers to its propensity to form thrombus and this is due to several factors, including strut thickness, strut width, strut shape, total scaffold surface area, scaffold pattern, scaffold length, scaffold diameter, surface roughness and surface chemistry. Some of these factors are interrelated. Low strut profile also leads to less neointimal proliferation as the neointima will proliferate to the degree necessary to cover the strut. As such coverage is a necessary step to complete healing. Thinner struts are believed to endothelialize and heal more rapidly.

Markers attached to a scaffold having thinner struts, however, may not hold as reliably as a scaffold having thicker struts since there is less surface contact area between the strut and marker. Embodiments of invention address this need.

According to another aspect a thickness of the combined marker and strut is kept below threshold values of about 150 microns while reliably retaining the marker in the hole.

According to other aspects of the invention, there is a scaffold, medical device, method for making such a scaffold, method of making a marker, attaching a marker to a strut, link or bar arm of a scaffold, or method for assembly of a medical device comprising such a scaffold having one or more, or any combination of the following things (1) through (26):
(1) A link, link element, strut or bar arm having a rectangular marker hole for holding a radiopaque marker. The link, link element, strut or bar arm being made from a polymer
(2) Marker side walls that are convex or concave.
(3) Hole side walls that are convex or concave.
(4) A tongue and groove connection between a marker side wall and a marker hole side wall. One of the marker and hole side wall provides the tongue and the other of the marker and hole side wall provides the tongue. The tongue is received within the groove.
(5) The polymer composition comprises poly(L-lactide).
(6) The marker according any of the embodiments shown in FIGS. 6-8.
(7) The marker having a plurality of flanges, such as the marker shown in FIG. 9.
(8) The radiopaque marker is comprised of platinum, platinum/iridium alloy, gold, iridium, tantalum, palladium, tungsten, niobium, zirconium, iron, zinc, tin, magnesium, manganese or their alloys.
(9) The marker inserted into the hole, with or without laterally restraining the marker when it is being forced into the marker hole.
(10) A method of inserting the marker into the hole wherein the hole is laterally restrained between members, e.g., a pair of bosses.
(11) A wall thickness for the link having the hole is between about 80 and about 100 microns, or between about 125 and about 160 microns.
(12) A link element is restrained by a rigid member or a member having at least 100 times the compressive stiffness of the link, e.g., steel or steel alloy, boss.
(13) After inserting the marker into the hole, the link is heated about 0-20 degrees above the scaffold polymer's Tg and prior to crimping, such as prior to and within 24 hours of crimping; wherein the heating improves a retention force maintaining a marker in a hole.
(14) A cold-forging or swaging process for attaching a marker to a scaffold, including deforming the marker between a first and second ram
(15) A marker hole that does not have a circular or elliptical opening before and/or after the marker is inserted into the hole.
(16) A marker hole that has a rectangular or square opening before and/or after the marker is inserted into the hole.
(17) A tongue and groove connection between the marker and side walls of the hole but not end walls of the hole.
(18) An upper and lower swaging surface used to force a marker into the hole; wherein a coefficient of friction is higher for the upper surface than the lower surface, or the coefficient of friction is about equal between the upper and lower surfaces.
(19) Side walls and/or end walls of the hole prior to inserting the marker into the hole are not curved, rounded, convex, or concave.
(20) The scaffold element having the marker hole has the same width, length and wall thickness as adjacent scaffold elements of the same type—e.g., links, struts or crowns—that do not have a marker hole; or the scaffold element having the marker hole has a width that is about 2 times higher than the width of the adjacent scaffold element of the same type.
(21) The scaffold element is a link connecting adjacent rings and the link extends parallel to a longitudinal axis of the scaffold.
(22) One or two rectangular markers are located on the same link.
(23) A method, comprising: using a radiopaque marker; using a polymer scaffold comprising an element having a hole formed in the element; and inserting the marker into the hole while laterally restraining the element.
(24) The method of (23) in combination with one or more, or any combination of items (a)-(i) in any combination:
   (a) wherein the marker is a rectangular marker and the inserting step re-shapes the marker to form a tongue and groove connection with walls of the hole.
   (b) wherein the marker is an X-shaped marker and the inserting step re-shapes the marker to form a tongue and groove connection with walls of the hole.
   (c) wherein the marker has a concave or convex sidewall surface and the inserting step re-shapes the marker to form a tongue and groove connection with walls of the hole.
   (d) wherein the concave or convex sidewall faces a wall of the element that extends parallel to a longitudinal axis of the element when placing the marker in the hole, and wherein the tongue and groove connection is formed with the wall of the element that extends parallel to the longitudinal axis.
   (e) wherein the marker is laterally restrained between members having a compressive stiffness that is at least 100 times higher than the compressive stiffness of the element, or the members are made from a material having a Young's modulus that is at least 100 times higher than the Young's modulus of the polymer.
   (f) wherein the scaffold is placed on a frame having a recess, and the element is placed within the recess to laterally restrain the element.
   (g) wherein the marker is inserted into the hole by cold forging or swaging.
   (h) wherein the element has a wall thickness of between 80 and 120 microns.
   (i) further including heating the scaffold after inserting the marker into the hole.
(25) A medical device, comprising: a scaffold made from a polymer, the scaffold including an element having a longitudinal axis and four sidewalls of a hole having a rectangular opening formed in the element; and a marker disposed within the hole; and a tongue and groove connection between at least two surfaces of the marker and respective adjacent walls of the hole.
(26) The medical device of (25) in combination with one or more, or any combination of items (a)-(i):
   (a) wherein two of the four sidewalls extend parallel to the longitudinal axis of the element and the tongue and groove connection is between surfaces of each of the two sidewalls and mating surfaces of the marker.
   (b) wherein a first two of the four sidewalls forms a tongue and groove connection with the marker and a second two sidewalls that extend perpendicular to the longitudinal axis do not form a tongue and groove connection.
   (c) wherein the tongue and groove connection comprises: a groove portion, comprising: medial portions of marker surfaces extending parallel to the longitudinal axis, the medial portions of marker surfaces being located distal top and bottom surfaces of the marker and proximal a centroid of the element, and top and bottom edges of the marker surfaces, the edges of the marker surfaces being proximal the upper and lower surfaces of the marker, respectively, and distal the centroid, wherein the medial portions of the marker surfaces are closer to each other than are the top and bottom edges of the marker surfaces, and a tongue portion, comprising: medial portions of sidewall surfaces extending parallel to the longitudinal axis, the medial portions of sidewall surfaces being located distal top and bottom surfaces of the element and proximal the centroid, and top and bottom edges of the sidewall surfaces, the edges being proximal the upper and lower surfaces of the sidewalls, respectively, and distal the centroid, wherein the medial portions of the sidewalls are closer to each other than are the top and bottom edges of the sidewall surfaces.
   (d) The medical device of Claim 14, wherein the medial portions of the marker surfaces are about 5%, about 3%, about 10%, 5-30% or 10-20% closer to each other than are the top and bottom edges of the marker surfaces.
   (e) wherein the tongue and groove connection comprises: a groove portion, comprising: medial portions of marker surfaces extending parallel to the longitudinal axis, the medial portions of marker surfaces being located distal top and bottom surfaces of the marker and proximal a centroid of the element, and top and bottom edges of the marker surfaces, the edges of the marker surfaces being proximal the upper and lower surfaces of the marker, respectively, and distal the centroid, wherein the medial portions of the marker surfaces are further from each other than are the top and bottom edges of the marker surfaces, and a tongue portion, comprising: medial portions of sidewall surfaces extending parallel to the longitudinal axis, the medial portions of sidewall surfaces being located distal top and bottom surfaces of the element and proximal the centroid, and top and bottom edges of the sidewall surfaces, the edges being proximal the upper and lower surfaces of the sidewalls, respectively, and distal the centroid, wherein the medial portions of the sidewalls are further from each other than are the top and bottom edges of the sidewall surfaces.
   (f) wherein the medial portions of the marker surfaces are about 5%, about 3%, about 10%, 5-30% or 10-20% further from each other than are the top and bottom edges of the marker surfaces.
   (g) wherein a top and bottom surface of the marker each have a recessed area.
   (h) wherein a length of the marker extending parallel to the longitudinal axis of the element is greater than a width thereof measured perpendicular to the longitudinal axis.
   (i) wherein a top and bottom surface of the marker protrudes from a top and bottom opening, respectively of the hole by about 0%, about 5% or about 10%.

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### BRIEF DESCRIPTION OF THE DRAWINGS

FIG. 1 is a perspective view of a portion of a prior art scaffold. The scaffold is shown in a crimped state (balloon not shown).

FIG. 2 is a top partial view of a prior art scaffold showing a link connecting adjacent rings. The link includes holes for holding radiopaque markers.

FIG. 3A is a partial side view of a scaffold having a link with a rectangle hole formed in a link element. The link element hole holds a marker. The scaffold is positioned over a frame configured to restrain the link when a marker is pressed into the hole.

FIG. 3B is a close-up view of the apparatus of FIG. 3A.

FIG. 3C is a cross-sectional view taken at Section IIIC-IIIC of FIG. 3B. This figure also shows a swaging surface of a ram head for pressing the marker into the hole.

FIG. 4A is a first cross-sectional view of a scaffold portion showing a first marker-type attached to a hole of a link element. The marker was attached using the apparatus described in FIGS. 3A-3C. In one embodiment the marker (prior to being pressed into the hole) had concave side walls. The left and right sides of the marker and wall make a tongue-and-groove connection with each other.

FIG. 4B is a second cross-sectional view of a scaffold portion showing a second marker-type attached to a hole of a link strut. The marker was attached using the apparatus described in FIGS. 3A-3C. In another embodiment the marker (prior to being pressed into the hole) had convex side walls. The left and right sides of the marker and wall also make a tongue-and-groove connection with each other.

FIGS. 5A and 5B are images of radiopaque markers pressed into a hole of a link while the side walls of the hole are restrained. These images are taken with an optical microscope. They are cross-sections through platinum markers that were installed in bioabsorbable scaffolds. They were prepared by mounting (encapsulating) the marker/scaffold assembly in epoxy and then grinding down to the plane of view using standard metallographic preparation methods (wetted silicon carbide abrasive papers of progressively finer grit size). The circular object is an air bubble entrapped within the hardened epoxy mounting material.

FIG. 6A is a perspective view of a rectangular marker not having any concave or convex side walls.

FIG. 6B is an approximate perspective view of the rectangle marker of FIG. 6A after inserted into a laterally restrained, rectangular marker hole using the apparatus of FIG. 3A. When walls of the link are restrained the marker side-walls form a convex shape, or the left and right sides of the marker and wall of the link strut (or element) make a tongue-and-groove connection with each other as shown in FIG. 4B.

FIG. 7A is a perspective view of an oval marker, or a rectangular marker formed with convex sidewalls.

FIG. 7B is an approximate perspective view of the marker of FIG. 7A after inserted into a laterally restrained, rectangular marker hole using the apparatus of FIG. 3A. When walls of the link are restrained the marker side-walls maintain the convex shape, or the left and right sides of the marker and wall of the link strut (or element) make a tongue-and-groove connection with each other as shown in FIG. 4B.

FIG. 8A is a perspective view of a rectangular marker formed with concave sidewalls.

FIG. 8B is an approximate perspective view of the marker of FIG. 8A after inserted into a laterally restrained, rectangular marker hole using the apparatus of FIG. 3A. When walls of the link are restrained the marker side-walls maintain the concave shape, or the left and right sides of the marker and wall of the link strut (or element) make a tongue-and-groove connection with each other as shown in FIG. 4A.

FIG. 9A is a perspective view of an X-shaped marker. This marker has four projecting flanges that extend radially outward from a center.

FIG. 9B is perspective view of the marker of FIG. 9A.

FIG. 10 is the same cross-sectional view taken at Section IIIC-IIIC of FIG. 3B, with the exception that the marker of FIG. 8A is replaced with the X-shaped marker of FIG. 9A-9B. This figure also shows a swaging surface of a ram head for pressing the marker into the hole.

FIG. 11 is a cross-sectional view of the scaffold with deformed X-shaped marker. The marker was preferably attached using the apparatus described in FIG. 10.

FIG. 12 is a perspective view of a mandrel and swaging arc comprising a ram head for swaging the markers as shown in FIGS. 11 of 3C.

### DETAILED DESCRIPTION

In the description like reference numbers appearing in the drawings and description designate corresponding or like elements among the different views.

For purposes of this disclosure, the following terms and definitions apply:

The terms “about,” “approximately,” “generally,” or “substantially” mean 30%, 20%, 15%, 10%, 5%, 4%, 3%, 2%, 1.5%, 1%, between 1-2%, 1-3%, 1-5%, or 0.5%-5% less or more than, less than, or more than a stated value, a range or each endpoint of a stated range, or a one-sigma, two-sigma, three-sigma variation from a stated mean or expected value (Gaussian distribution). For example, d1 about d2 means d1 is 30%, 20%, 15%, 10%, 5%, 4%, 3%, 2%, 1.5%, 1%, 0% or between 1-2%, 1-3%, 1-5%, or 0.5%-5% different from d2. If d1 is a mean value, then d2 is about d1 means d2 is within a one-sigma, two-sigma, or three-sigma variance or standard deviation from d1.

It is understood that any numerical value, range, or either range endpoint (including, e.g., “approximately none”, “about none”, “about all”, etc.) preceded by the word “about,” “approximately,” “generally,” or “substantially” in this disclosure also describes or discloses the same numerical value, range, or either range endpoint not preceded by the word “about,” “approximately,” “generally,” or “substantially.”

A “stent” means a permanent, durable or non-degrading structure, usually comprised of a non-degrading metal or metal alloy structure, generally speaking, while a “scaffold” means a temporary structure comprising a bioresorbable or biodegradable polymer, metal, alloy or combination thereof and capable of radially supporting a vessel for a limited period of time, e.g., 3, 6 or 12 months following implantation. It is understood, however, that the art sometimes uses the term “stent” when referring to either type of structure.

“Inflated diameter” or “expanded diameter” refers to the inner diameter or the outer diameter the scaffold attains when its supporting balloon is inflated to expand the scaffold from its crimped configuration to implant the scaffold within a vessel. The inflated diameter may refer to a post-dilation balloon diameter which is beyond the nominal balloon diameter, e.g., a 6.5 mm balloon (i.e., a balloon having a 6.5 mm nominal diameter when inflated to a nominal balloon pressure such as 6 times atmospheric pressure) has about a 7.4 mm post-dilation diameter, or a 6.0 mm balloon has about a 6.5 mm post-dilation diameter. The nominal to post dilation ratios for a balloon may range from 1.05 to 1.15 (i.e., a post-dilation diameter may be 5% to 15% greater than a nominal inflated balloon diameter). The scaffold diameter, after attaining an inflated diameter by balloon pressure, will to some degree decrease in diameter due to recoil effects related primarily to, any or all of, the manner in which the scaffold was fabricated and processed, the scaffold material and the scaffold design.

When reference is made to a diameter it shall mean the inner diameter or the outer diameter, unless stated or implied otherwise given the context of the description.

When reference is made to a scaffold strut, it also applies to a link or bar arm.

“Post-dilation diameter” (PDD) of a scaffold refers to the inner diameter of the scaffold after being increased to its expanded diameter and the balloon removed from the patient's vasculature. The PDD accounts for the effects of recoil. For example, an acute PDD refers to the scaffold diameter that accounts for an acute recoil in the scaffold.

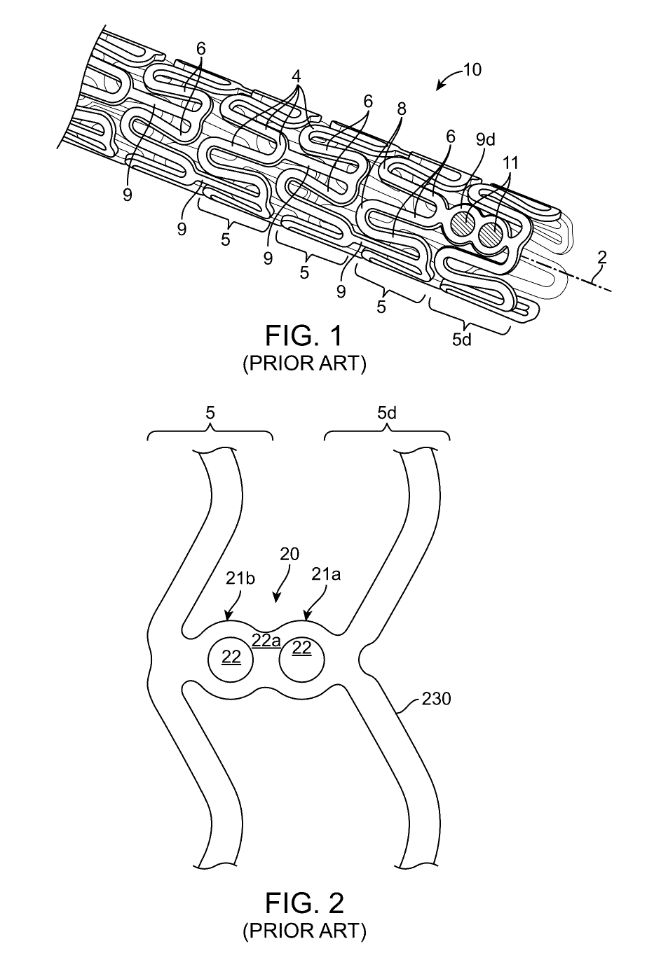

A “before-crimp diameter” means an outer diameter (OD) of a tube from which the scaffold was made (e.g., the scaffold is cut from a dip coated, injection molded, extruded, radially expanded, die drawn, and /or annealed tube) or the scaffold before it is crimped to a balloon. Similarly, a “crimped diameter” means the OD of the scaffold when crimped to a balloon. The “before-crimp diameter” can be about 2 to 2.5, 2 to 2.3, 2.3, 2, 2.5, 3.0 times greater than the crimped diameter and about 0.9, 1.0, 1.1, 1.3 and about 1-1.5 times higher than an expanded diameter, the nominal balloon diameter, or post-dilation diameter. Crimping, for purposes of this disclosure, means a diameter reduction of a scaffold characterized by a significant plastic deformation, i.e., more than 10%, or more than 50% of the diameter reduction is attributed to plastic deformation, such as at a crown in the case of a stent or scaffold that has an undulating ring pattern, e.g., FIG. 1. When the scaffold is deployed or expanded by the balloon, the inflated balloon plastically deforms the scaffold from its crimped diameter. Methods for crimping scaffolds made according to the disclosure are described in US20130255853 (attorney docket 62571.628).

A material “comprising” or “comprises” poly(L-lactide) or PLLA includes, but is not limited to, a PLLA polymer, a blend or mixture including PLLA and another polymer, and a copolymer of PLLA and another polymer. Thus, a strut comprising PLLA means the strut may be made from a material including any of a PLLA polymer, a blend or mixture including PLLA and another polymer, and a copolymer of PLLA and another polymer.

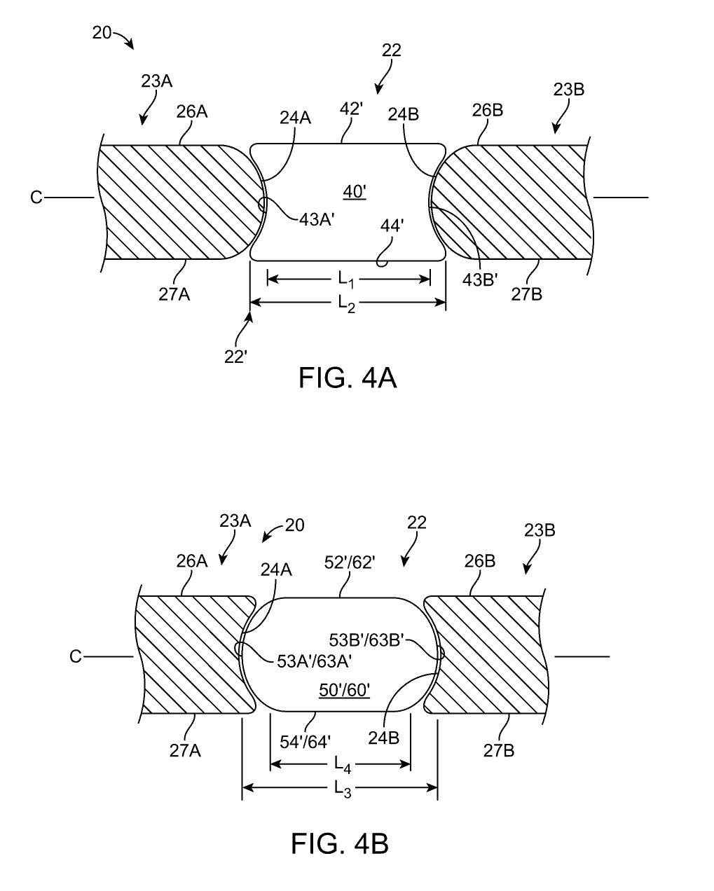

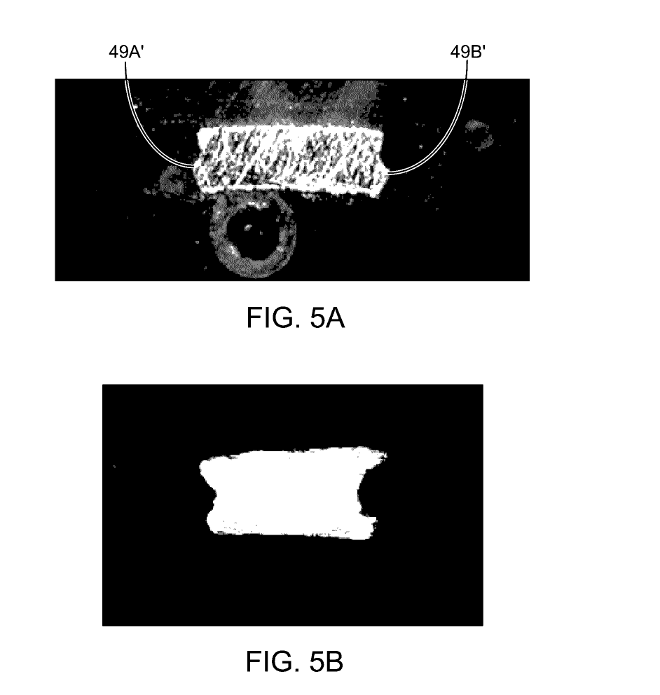

An undeformed, deformed or swaged marker, or a hole of a scaffold link element has a “convex” or “concave” sidewall surface when the average curvature of the hole side wall or marker side wall is substantially convex or concave, respectively. For example, the marker shown in FIG. 4A has concave side walls 43A′ and 43B′ whereas the side walls 24A and 24B of the hole 22 have a convex surface. The marker shown in FIG. 5C also has a curvature that is concave because the side walls are substantially concave.

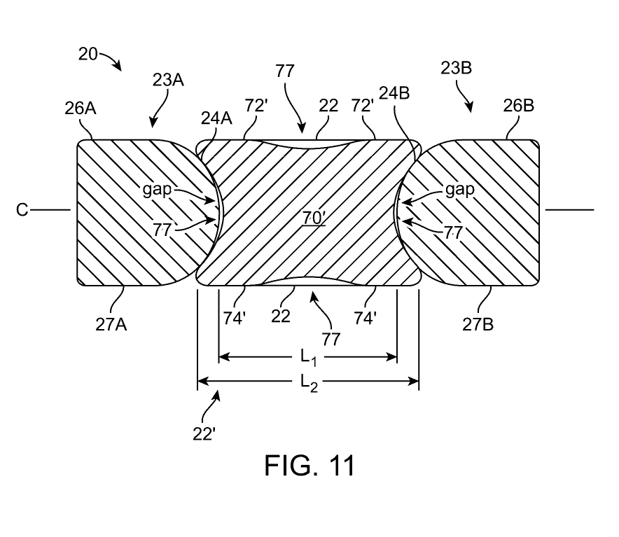

A marker inserted into a hole of a scaffold link element (or strut) forms a “tongue-and-groove” or “tongue/groove” connection with the hole when a medial portion of the wall of the marker or adjoining wall of the (link element or strut) hole extends into the adjoining medial portion of the wall of the hole or marker, respectively. For example, the medial portions (between the upper and lower edges) of the hole walls 24A, 24B in FIG. 4A represent tongue parts of the tongue/groove connection whereas the medial portions of the marker wall 43A′, 43B′ (between the upper and lower edges) of the marker 40′, 24B in FIG. 4A represent groove parts. Examples of tongue-and-groove attachments or connections between marker and hole walls are shown in FIGS. 5C, 5B, 4A, 4B and 11. As shown in FIGS. 4A and 11 the medial extent (L1) is less than the extent between upper and lower edges (L2), and in FIG. 4B the medial extent (L3) is greater than the extent between upper and lower edges (L4).

A “lateral restraint” or “laterally restraining” means a physical or mechanical restraint or restraining of a scaffold element (of a scaffold element such as a link, strut or crown having a marker hole) that prevents or resists the width of the element from changing when a radiopaque marker is being forced into a marker hole formed in the element during a swaging or forging process. Without the restraint side walls of the element will bulge laterally outward to accommodate the marker. The direction of bulging is circumferentially in respect to the circumferential direction of a tubular scaffold body.

Bioresorbable scaffolds comprised of biodegradable polyester polymers are radiolucent. In order to provide for fluoroscopic visualization, radiopaque markers are placed on the scaffold. For example, the scaffold described in U.S. Pat. No. 8,388,673 ('673 patent) has two platinum markers 206 secured at each end of the scaffold 200, as shown in FIG. 2 of the '673 patent.

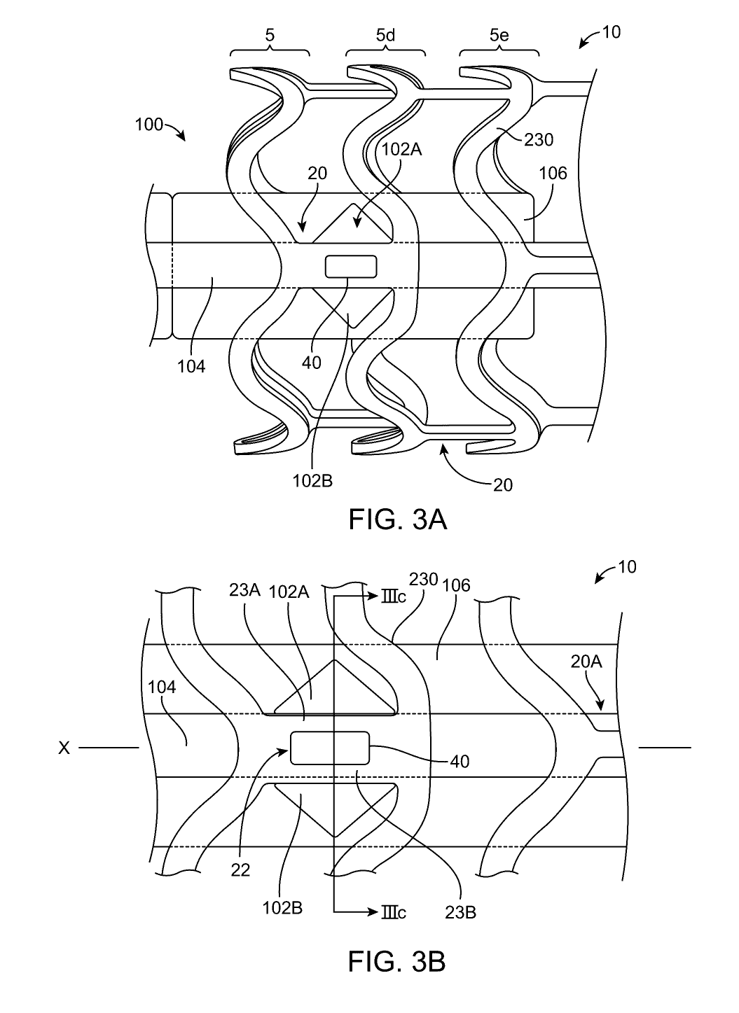

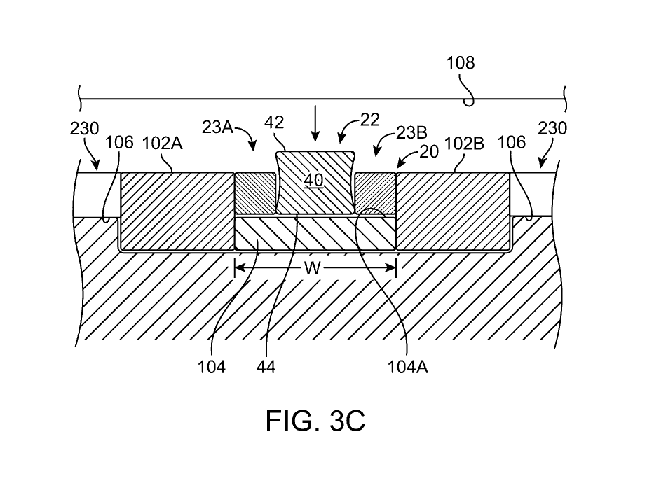

FIG. 2 is a top planar view of a portion of a polymer scaffold, e.g., a polymer scaffold having a pattern of rings interconnected by links as in the case of the '673 patent embodiments. There is a link element or link 20 extending between rings 5d, 5 in FIG. 2. The link 20 has formed left and right structures or strut portions 21b, 21a, respectively, for holding a radiopaque marker. The markers are retainable in holes 22 formed by the structures 21a, 21b. The surface 22a corresponds to an abluminal surface of the scaffold. An example of a corresponding scaffold structure having the link 20 is described in FIGS. 2, 5A-5D, 6A-6E and col. 9, line 3 through col. 14, line 17 of the '673 patent. The embodiments of a scaffold having a marker-holding link structure or method for making the same according to this disclosure in some embodiments include the embodiments of a scaffold pattern according to FIGS. 2, 5A-5D, 6A-6E and col. 9, line 3 through col. 14, line 17 of the '673 patent, with the exception of the hole shape being rectangular (rather than circular) and link construction being rectangular, as shown in FIG. 3A-3B. The embodiments of a scaffold having a marker-holding link structure or method for making the same according to this disclosure in some embodiments include any of the embodiments described in FIGS. 2, 3, 4, 5A, 5B, 6A, 6B, 9A and 9B and the accompanying paragraphs [0130]-[0143], [0171]-[0175] of US20110190871 ('871 Pub).

Additional scaffold structure considered within the scope of this disclosure is the alternative scaffold patterns having the marker structure for receiving markers as described in FIGS. 11A, 11B and 11E and the accompanying description in paragraphs [0177]-[0180] of the '871 Pub. In these embodiments the values D0, D1 and D2 would apply to the relevant structure surrounding the holes 512, 518 and 534 shown in the '871 Pub., as will be readily understood.

In the discussion that follows when the same element numbering is used the same description applies, except in those circumstances where it is readily apparent that the identical description does not apply. Also, reference is made to a radiopaque marker and marker hole before and after inserting the marker into the marker hole using a swaging process. The deformed vs. undeformed markers are distinguished by use of the prime symbol. Thus, for example, a marker undeformed is marker 40, whereas the same marker in a deformed state is marker 40′. In the examples below the marker is inserted into a link 20 of a scaffold. However, the disclosure is not limited to a link or link element adapted to receive radiopaque markers. The marker and methods for insertion according to the disclosure equally applies to markers inserted into struts, crowns, or other scaffold structure capable of having a rectangular marker hole formed therein without departing from the scope of invention.

FIGS. 3A and 3B show a portion of a swaging apparatus disposed within a scaffold 10. The device has a recess positioned to receive a link 20 that has a rectangle marker hole 22 for holding a radiopaque marker, e.g., a radiopaque marker 40 also shown in FIG. 8A. The link 20 has a longitudinal axis (x-axis in FIG. 3A). The marker hole has four sidewalls: two side walls that extend parallel to the link's longitudinal axis and two end walls that extend perpendicular to this axis.

The apparatus is elongate, extending along the longitudinal axis of the scaffold and includes a frame 106 holding members 102A, 102B and a strip 104. Preferably these members are bosses, and more preferably triangular bosses that have a length about equal to the length of the link 20 and adjacent rings 5. The bosses are made of a relatively rigid material, such as a steel or alloy of steel, stainless steel, tool steel or tungsten carbide. The effective compressive stiffness of the bosses (i.e., when fixed in the recesses and opposing outward movement of the side walls of the link 20) may be 100 times, 1000 times or at least 100 times higher than the compressive or flexural stiffness of the link 20 side walls.

The apparatus is used to restrain the link 20 while the radiopaque marker 40 is pressed into the rectangular hole 22. FIG. 3C is a cross-sectional view of the apparatus and scaffold 10 taken at Section IIIC-IIIC of FIG. 3B and additionally showing a ram or swaging head 108 used to force the marker 40 into the hole 22 so that top and bottom surfaces become flush with the abluminal and luminal surfaces of the link element 22.

The marker 40 shown in FIGS. 3A-3C is a rectangular marker 40 having concave sidewalls, an example of which is shown in FIG. 8A. Shown is a perspective view of the marker 40 before being pressed into the hole 22. The deformed marker 40′ is shown in FIG. 8B, which is approximately the shape taken when the marker surfaces are flush with the surfaces of element 22. The concave side walls 43A, 43B and 43A′, 43B′ are shown in FIGS. 8A and 8B, respectively. The marker 40 has a top surface 42 and a bottom surface 44. The end walls extending between the concave walls 43A, 43B may be flat.

Referring again to FIGS. 3A, 3B and 3C the apparatus is arranged so that the link 20 may be snugly received between members 102A, 102B and rests on a top surface 104A of the strip 104. The bottom surface 44 of the marker 40 rests on the surface 104A and the top surface 42 faces the swaging head 108, which is pressed onto the surface 42 to force the marker surface 42 flush with the top surface of the link 20. The marker hole 22 is positioned between the members 102A, 102B. As mentioned above, the members may be made from a relatively rigid material to effective prevent any lateral deflection of the link side walls. The members 102A, 102B may be positioned within a recessed area of the frame 106 and spaced apart so that the space between the two members snugly holds the link; that is, the distance between the members is “w” or about the width of link 20. The frame and members 102 provide a lateral restraint of the link 20 side walls when the rectangular marker 40 is forced into the hole by the head 108 and opposing strip 104. The restraint applies in the circumferential direction or preventing movement or bulging of the link 20 left or right in FIG. 3C, or bulging in the scaffold's circumferential direction, which is perpendicular to the x-axis in FIG. 3B.

This lateral restraint provided by the members and frame is intended to force a tongue/groove connection between the deformed marker and walls of the marker hole 22, or alternatively form a concave/convex engagement between surfaces of the hole and marker. In the preferred embodiment, to achieve either of these results, the marker 40 is arranged so that its concave walls 43A and 43B face the respective members 102A and 102B. The head 108 is brought down to engage the top surface 42. The head 108 continues to press down until both surfaces 42, 44 of the deformable marker 40 are flush or nearly flush with the upper/abluminal and lower/luminal surfaces of the link 20.

In other embodiments a lateral restraint to outward bulging may be provided by increasing the wall thickness or width of the link, thereby effectively increasing the flexural rigidity of the link's side walls. According to these embodiments a rectangular hole receives a rectangular marker without applying a mechanical or physical restraint, but which may also produce the desired tongue and groove connection by virtue of the link element having relatively high wall stiffness.

In other embodiments the link wall may be made more thick, which has the effect of increasing the frictional force for holding the marker in the rectangular hole. The rectangular hole may have a thickness of between about 125 microns and about 160 microns. It has been found that within this range of wall thickness there is an acceptable level of retention force (due to the added friction) even in those cases where there is no significant, or no tongue/groove connection present.

FIG. 4A is a cross-sectional view of the hole 22 and marker 40′ after the marker is pressed between the surfaces 104A and 108 using the arrangement shown in FIG. 3C. The surface 42′ is substantially flush with the surfaces 26A, 26B of the hole 22. Additionally, the hole wall 24A and marker wall 43A′, and the hole wall 24B and marker wall 43B′ form respective left and right tongue/groove connections and may also have concave/convex surfaces to retain the marker 40′ within the hole 22. A medial portion of the marker 40′ has a width (L1) measured between the left and right surfaces 43A′, 43B′. The width measured between the left and right sides of the upper and/or lower edges (L2) is greater than L1. Stated differently, a portion of each of the wall surfaces 43A′, 43B′ distal of the top surface 42′ and lower surface 44′ and proximal the centerline “C” passing through the marker 40′ in FIG. 4A (the line “C” may be thought of as the geometric centerline of the link 20 or location of the centroid of the link 20) is the location on the surfaces 43A′ and 43B′ for measurement L1, and a portion of each of the wall surfaces 43A′, 43B′ proximal the top surface 42′ and lower surface 44′ and distal the measurement location for L1 is the measurement location for L2 (for those circumstances where the measured distance between top edges is different form the measured distance between bottom edges are not equal, L2 is the average or greater distance between the two). According to the embodiments L1 ranges from 2-5%, 5-10%, or 5-30% less than L2. In tests these ranges were achieved for a link 20 thickness (or wall thickness) of about 100 microns.

The walls of the marker 40′ are concave, as mentioned above. The corresponding walls 24A, 24B of the hole 22 may be deformed into convex shapes, and/or may only form a tongue portion of a tongue/groove connection. Surfaces of the left and right sides of the hole's deformed side walls 24A, 24B proximal centerline C and distal the top and lower surfaces are closer to each other than are the surfaces proximal the upper and lower edges and distal the centerline C.

In some embodiments the concave marker shape may be maintained, while in other embodiments the concave shape is altered, yet there is retained the tongue/groove connection. In some embodiments a marker has concave sidewalls and after being pressed into a hole the marker sidewalls may form the tongue or groove portion of the tongue/groove connection.

FIGS. 5A-5B are two microscopic images of metallographically prepared cross-sections of markers attached to struts of a scaffold.

In FIG. 5B the concave shape of marker 40 is retained and the tongue/groove connection is present, whereas in FIG. 5A the concave shape is no longer present; however, there is still a tongue and groove connection where the deformed marker forms a tongue portion 49A′ and 49B′ (as opposed to a groove, as in FIG. 5B). In this example the marker began as marker 40. When it was pressed into the link 20 however it changed its shape to cause it to lose its concave surface. Additionally, the marker does not provide the groove part of the tongue/groove connection (in contrast to the marker shown in FIG. 5B). Instead, the marker provides the tongue part, as indicated at 49A′ and 49B′ in the image.

The shape of the marker may be affected by using swaging or forging heads with differing coefficients of friction. For example, using an upper swaging head with higher coefficient of friction than the opposing bottom swaging surface can produce greater lateral flow near the bottom surface.

Referring again to FIGS. 8A-8B, the marker 40 may be made in a variety of ways. Starting with a round wire of radiopaque material, the wire is first flattened to produce a wire having a shape like that shown for marker 50 in FIG. 7A. Next, concave sides may be made by either drawing the flattened wire through a die or using rollers. After making the concave sides the wire is cut into the desired lengths. Alternatively, the marker 40 may be made by stamping and coining, powder metallurgy (compaction followed by sintering) or by a 3D printing technique. The marker 40 may be between 10%-30% thicker than the link's wall thickness (as shown in FIG. 3C the undeformed marker 40 is about 20-30% thicker than the wall thickness of the link 20).

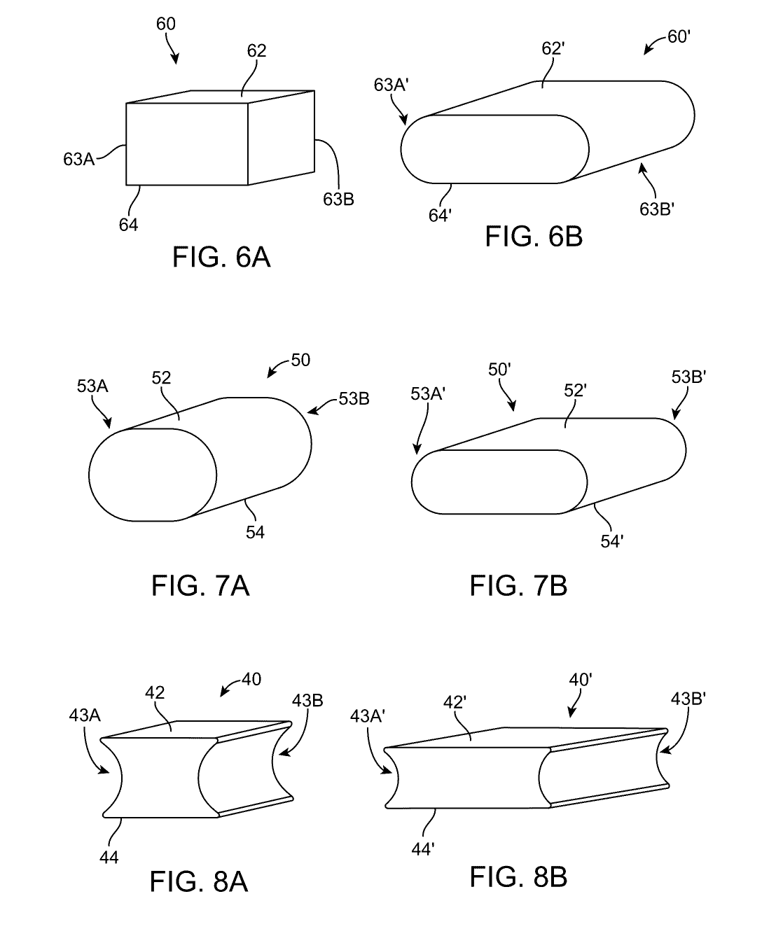

FIGS. 6A-6B show an alternative embodiment of a marker 60 configured for being inserted into a marker hole to make a tongue/groove connection using, e.g., the apparatus of FIGS. 3A-3C. Marker 60 has all flat sides, and flat top and bottom surfaces. In the case of everywhere equal length sides for marker 60 this rectangular marker may be regarded as a cube. The marker 40 may be between 10%-30% thicker than the link's wall thickness. When placed in the marker hole this marker takes on a shape similar to marker 60′, which has a top surface 62′, bottom surface 64′ and convex side walls 63A′ and 63B′.

FIGS. 7A-7B show an alternative embodiment of a marker 50 configured for being inserted into a marker hole to make a tongue/groove connection using, e.g., the apparatus of FIGS. 3A-3C. Marker 50 has two flat sides and two curved sides, as in the case of marker 40. However, in this case marker 50 is made with convex, as opposed to concave sides. The marker 50 may be between 10%-30% thicker than the link's wall thickness. The end walls extending between the convex side walls 53A, 53B may be flat. When placed in the marker hole this marker takes on a shape similar to marker 50′, which has a top surface 52′, bottom surface 54′ and convex side walls 53A′ and 53B′.

FIG. 4B is a cross-sectional view of the link strut hole 22 and marker 50′/60′ after the marker is pressed between the surfaces 104 and 108 using the arrangement shown in FIG. 3C. The surface 62′/52′ is substantially flush with the surfaces 26A, 26B of the hole 22. Additionally, the hole wall 24A and marker wall 63A′/53A′, and the hole wall 24B and marker wall 63B′/53B′ each form left and right tongue/groove connections to retain the marker 60′/50′ within the hole 22′. A medial portion of the marker 50′/60′ has a width L3 measured between the left and right surfaces 63A′/53A′, 63A′/53B′. The width between the left and right upper and/or lower edges L4 is less than the width L3. Stated differently, a portion of each of the wall surfaces 53A′/63A′, 53B′/63B′ distal of the top surface 52′/62′ and lower surface 54′/64′ and proximal the centerline “C” passing through the marker 40′ In FIG. 4B (the line “C” may be thought of as the geometric centerline of the link 20 or location of the centroid of the link 20) is the location on the surfaces 53A′/63A′ and 53B′/63B′ for measurement L3, and a portion of each of the wall surfaces 53A′/63A′, 53B′/63B′ proximal the top surface 52′/62′ and lower surface 54′/64′ and distal the measurement location L3 is the measurement location L4. According to the embodiments L4 ranges from less than L3 (when the measurement between an upper edge is different from the length between a lower edge, L4 is the average or larger distance between the two). In tests these ranges were achieved for a link 20 thickness (or wall thickness) of about 100 microns.

The walls of the marker 50′/60′ are convex, as mentioned above. The corresponding walls 24A, 24B of the hole 22 may be deformed into concave shapes, or may only form the groove portion of a tongue/groove connection. Surfaces of the left and right sides of the hole wall 24A, 24B proximal centerline C and distal the top and lower surfaces are further apart from each other than are the surfaces proximal the upper and lower surface and distal the centerline C.

In some embodiments the convex marker shape may be maintained, while in other embodiments the convex shape is altered, yet there is retained the tongue/groove connection. In some embodiments a marker has convex sidewalls and after being pressed into a hole the marker sidewalls may form the tongue or groove portion of the tongue/groove connection.

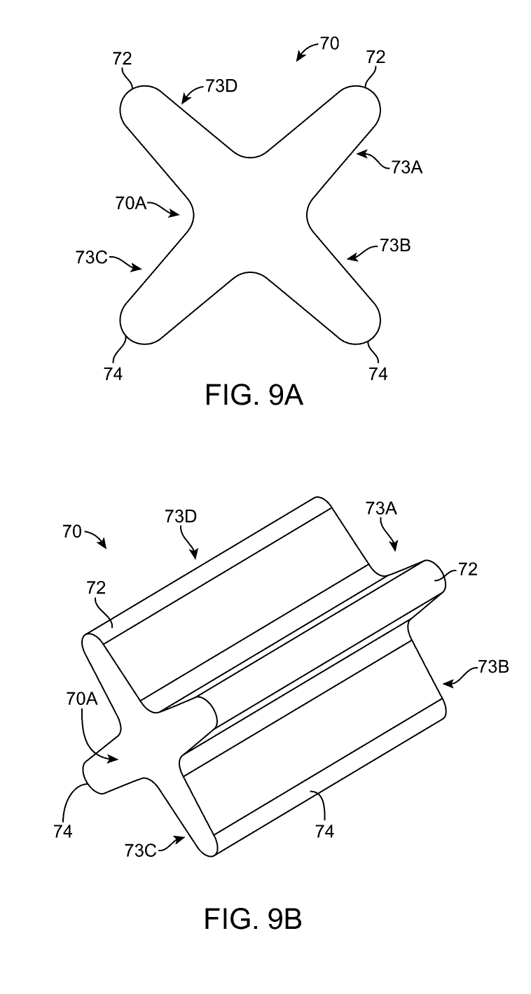

Referring to FIGS. 9A-9B there is shown front and perspective views, respectively, of an X-shaped radiopaque marker 70. This marker has four projecting flanges 73A, 73B, 73C and 73D that extend radially outward from a center 70A. The flanges 73A and 73D form surfaces 72 and the flanges 73B and 73C form surfaces 74. Each of the flanges may have a constant thickness from center 70A to the tips (where surfaces 72, 74 are located) or they may taper from center 70A to the tips. The marker cross section may be formed from wire of radiopaque material by drawing through a die or progression of dies, or by roll-forming.

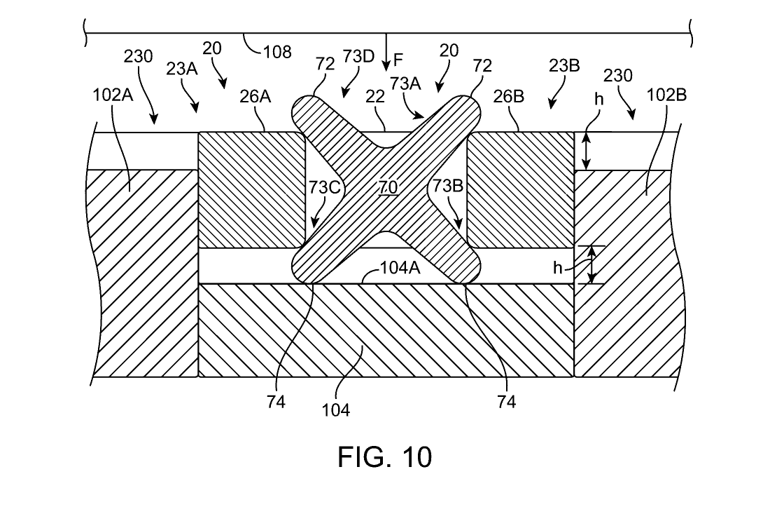

Referring to FIG. 10, marker 70 is disposed within hole 22 of link 20, which is received between the members 102A, 102B of the apparatus discussed earlier in connection with FIGS. 3A-3C. FIG. 10 is a cross-sectional view taken at Section IIIC-IIIC, like FIG. 3C, except that in this case marker 40 is replaced with marker 70. Additionally, link 20 is raised off the surface 104A by the amount “h” due to the legs 73C, 73D extending out of the bottom opening of the hole 22. Surfaces 74 contact the surface 104A and the surfaces 72 of the flanges 73A and 73D are arranged to contact the head 108.

During the Swaging process the head is brought down upon the surfaces 72 and continues pressing downward until the head 108 comes into contact with the top surfaces of the members 102A, 102B. The process is preferably down at room or ambient temperatures thus it may be regarded as a cold-forging process (the same cold-forging process applies when markers 40, 50 and 60 are swaged using the apparatus).

FIG. 11 shows the finished product, which is the marker 70′ inserted into the hole 22 with surfaces 72′ and 74′ about flush with the respective surfaces 26A, 26B and 27B, 27B. There is also formed a tongue/groove connection for marker 70′ and hole 22. The hole 22 as shown has convex side walls 24A, 24B and the marker 70′ has what approximates concave sidewalls 73A′ and 73B′ resulting from the deformation of the X-shape into the rectangular shape having recesses 77 along the top surface 72′, bottom surface 74′ and walls 73A′ and 73B′. The recesses 77 along the sidewalls 73A′ and 73B′ provide a groove for the tongue/groove connection with the respective walls 24A, 24B.

As compared to the rectangular-like markers of 50, 60 and 40 the deformed X-shape marker is forced into the rectangular shape of the hole by plastic deformation of the flanges 73A-73D. Additionally, the material will not completely flow to re-form the X-shaped marker into a rectangular form like that of marker 40-60. This is indicated by the marker 70′ having the recess 77 at the top surface 72′, bottom surface 74′. Additionally, in some embodiments it is expected there will be a gap between the side walls 73A′, 73B′ and side walls 24A, 24B respectively, near the centerline C of the link 20. The material between a side recess and top (or bottom) recess represents the material from a respective flange (compare FIG. 10 with FIG. 11) that flowed when the marker was forced into the hole 22.

A medial portion of the marker 70′ has a width (L1) measured between the left and right surfaces 73A′, 73B′. The width measured between the left and right sides of the upper and/or lower edges (L2) is greater than L1. Stated differently, a portion of each of the wall surfaces 73A′, 73B′ distal of the top surface 72′ and lower surface 74′ and proximal the centerline “C” passing through the marker 70′ in FIG. 4A (the line “C” may be thought of as the geometric centerline of the link 20 or location of the centroid of the link 20) is the location on the surfaces 73A′ and 73B′ for measurement L1, and a portion of each of the wall surfaces 73A′, 73B′ proximal the top surface 72′ and lower surface 74′ and distal the measurement location for L1 is the measurement location for L2 (for those circumstances where the measured distance between top edges is different from the measured distance between bottom edges are not equal, L2 is the average or greater distance between the two). According to the embodiments L1 ranges from 2-5%, 5-10%, 10-20%, 20-30% or 5-30% less than L2.

The walls of the marker 70′ are concave, as mentioned above. The corresponding walls 24A, 24B of the hole 22 may be deformed into convex shapes, and/or may only form a tongue portion of a tongue/groove connection. Surfaces of the left and right sides of the hole's deformed side walls 24A, 24B proximal centerline C and distal the top and lower surfaces are closer to each other than are the surfaces proximal the upper and lower edges and distal the centerline C.

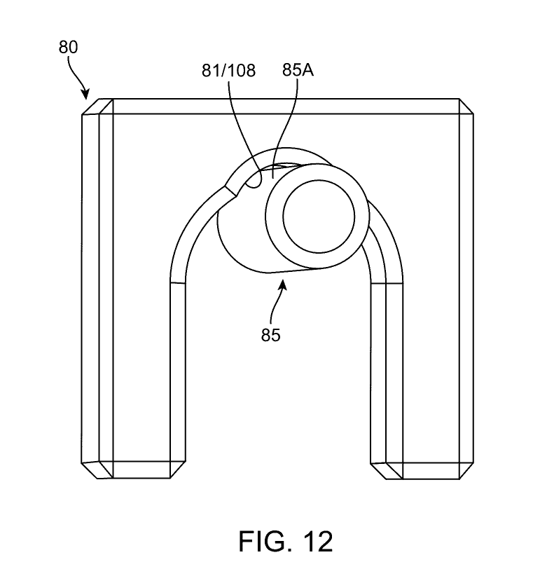

FIG. 12 shows an embodiment of a swaging arc 80 that may be used to forge or swage the marker 40, 50, 60 or 70 into the marker hole 20 as discussed above. The swaging arc 80 includes a rounded forging head 81 that may be used as the forging surface 108 shown in FIGS. 3A and 10. The scaffold may be received on the surface 85A of a mandrel 85. The frame 106 described earlier in connection with FIGS. 3A-3C may be formed on a top surface of the mandrel 85.

According to another aspect of the disclosure there is a heating step for a scaffold following marker placement. In some embodiments this heating step may correspond to a rejuvenation step of the scaffold polymer, prior to crimping, to remove aging effects of the polymer.

Thermal rejuvenation (including thermal treatment of a bioresorbable scaffold above Tg, but below melting temperature (Tm) of the polymer scaffold) prior to a crimping process may reverse or remove the physical ageing of a polymeric scaffold, which may reduce crimping damage (e.g., at the crests of a scaffold) and/or instances of dislodgment of a marker.

According to some embodiments a scaffold is thermally treated, mechanically strained, or solvent treated to induce a rejuvenation or erasure of ageing in a polymer shortly before crimping the scaffold to a balloon and after marker placement. Rejuvenation erases or reverses changes in physical properties caused by physical ageing by returning the polymer to a less aged or even an un-aged state. Physical ageing causes the polymer to move toward a thermodynamic equilibrium state, while rejuvenation moves the material away from thermodynamic equilibrium. Therefore, rejuvenation may modify properties of a polymer in a direction opposite to that caused by physical ageing. For example, rejuvenation may decrease density (increase specific volume) of the polymer, increase elongation at break of the polymer, decrease modulus of the polymer, increase enthalpy, or any combination thereof.

According to some embodiments, rejuvenation is desired for reversal or erasure of physical ageing of a polymer that was previously processed. Rejuvenation is not however intended to remove, reverse, or erase memory of previous processing steps. Therefore, rejuvenation also does not educate or impart memory to a scaffold or tube. Memory may refer to transient polymer chain structure and transient polymer properties provided by previous processing steps. This includes processing steps that radially strengthen a tube from which a scaffold is formed by inducing a biaxial orientation of polymer chains in the tube as described herein.

In reference to a marker—scaffold integrity or resistance to dislodgment during crimping, it has been found that a heating step can help reduce instances where crimping causes dislodgment of a marker. According to some embodiments, any of the foregoing embodiments for a marker held within the scaffold hole 22 can include, after the marker has been placed in the hole, a heating step shortly before crimping, e.g., within 24 hours of crimping. It has been found that the scaffold is better able to retain the marker in the hole 22 following heating. A mechanical strain, e.g. a limited radial expansion, or thermal rejuvenation (raise the scaffold temperature above the glass transition temperature (Tg) of the load-bearing portion of the scaffold polymer for a brief time period) can have a beneficial effect on scaffold structural integrity following crimping and/or after balloon expansion from a crimped state.

In particular, these strain-inducing processes tend to beneficially affect the hole 22 dimensions surrounding the marker when the hole is deformed in the manner discussed earlier in connection with FIGS. 4A, 4B and 11.

According to some embodiments the scaffold after marker placement is heated to about 20 degrees, or 30 degrees above the glass transition temperature of the polymer for a period of between 10-20 minutes; more preferably the scaffold load bearing structure (e.g., the portion made from a polymer tube or sheet of material) is a polymer comprising poly(L-lactide) and its temperature is raised to between about 80 and 85 Deg. C for 10-20 minutes following marker placement.

According to some embodiments it has been found that raising the temperature of the scaffold after marker placement re-shapes portions of the hole 22 to improve the fit of the marker with the hole, especially for marker 70. With reference to FIG. 11 after the marker 70 is placed in the hole 22 there may exist gaps between the sidewalls, as discussed earlier, that can be removed or filled with wall material after heating. Additionally, with respect to any of the embodiments of FIGS. 4A, 4B and 11 the hole shape deforms to produce a lip or edge, which may produce a higher resistance to dislodgment than for a scaffold-marker structure not subsequently treated by a rejuvenation step.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in claims should not be construed to limit the invention to the specific embodiments disclosed in the specification.

What is claimed is:

1. A method, comprising:
using a radiopaque marker;
using a polymer scaffold comprising an element having a hole formed in the element; and
inserting the marker into the hole while laterally restraining the element.

2. The method of claim 1, wherein the marker is a rectangular marker and the inserting step re-shapes the marker to form a tongue and groove connection with walls of the hole.

3. The method of claim 1, wherein the marker is an X-shaped marker and the inserting step re-shapes the marker to form a tongue and groove connection with walls of the hole.

4. The method of claim 1, wherein the marker has a concave or convex sidewall surface and the inserting step re-shapes the marker to form a tongue and groove connection with walls of the hole.

5. The method of claim 4, wherein the concave or convex sidewall faces a wall of the element that extends parallel to a longitudinal axis of the element when placing the marker in the hole, and wherein the tongue and groove connection is formed with the wall of the element that extends parallel to the longitudinal axis.

6. The method of claim 1, wherein the marker is laterally restrained between members having a compressive stiffness that is at least 100 times higher than the compressive stiffness of the element, or the members are made from a material having a Young's modulus that is at least 100 times higher than the Young's modulus of the polymer.

7. The method of claim 1, wherein the scaffold is placed on a frame having a recess, and the element is placed within the recess to laterally restrain the element.

8. The method of claim 1, wherein the marker is inserted into the hole by cold forging or swaging.

9. The method of claim 1, wherein the element has a wall thickness of between 80 and 120 microns.

10. The method of claim 1, further including heating the scaffold after inserting the marker into the hole.

11. A medical device, comprising:
a scaffold made from a polymer, the scaffold including an element having a longitudinal axis and four sidewalls of a hole having a rectangular opening formed in the element; and
a marker disposed within the hole; and
a tongue and groove connection between at least two surfaces of the marker and respective adjacent walls of the hole.

12. The medical device of claim 11, wherein two of the four sidewalls extend parallel to the longitudinal axis of the element and the tongue and groove connection is between surfaces of each of the two sidewalls and mating surfaces of the marker.

13. The medical device of claim 12, wherein a first two of the four sidewalls forms a tongue and groove connection with the marker and a second two sidewalls that extend perpendicular to the longitudinal axis do not form a tongue and groove connection.

14. The medical device of claim 11, wherein the tongue and groove connection comprises:
a groove portion, comprising:
medial portions of marker surfaces extending parallel to the longitudinal axis, the medial portions of marker surfaces being located distal top and bottom surfaces of the marker and proximal a centroid of the element, and
top and bottom edges of the marker surfaces, the edges of the marker surfaces being proximal the upper and lower surfaces of the marker, respectively, and distal the centroid,
wherein the medial portions of the marker surfaces are closer to each other than are the top and bottom edges of the marker surfaces, and
a tongue portion, comprising:
medial portions of sidewall surfaces extending parallel to the longitudinal axis, the medial portions of sidewall surfaces being located distal top and bottom surfaces of the element and proximal the centroid, and
top and bottom edges of the sidewall surfaces, the edges being proximal the upper and lower surfaces of the sidewalls, respectively, and distal the centroid,
wherein the medial portions of the sidewalls are closer to each other than are the top and bottom edges of the sidewall surfaces.

15. The medical device of claim 14, wherein the medial portions of the marker surfaces are about 5%, about 3%, about 10%, 5-30% or 10-20% closer to each other than are the top and bottom edges of the marker surfaces.

16. The medical device of claim 11, wherein the tongue and groove connection comprises:
a groove portion, comprising:
medial portions of marker surfaces extending parallel to the longitudinal axis, the medial portions of marker surfaces being located distal top and bottom surfaces of the marker and proximal a centroid of the element, and
top and bottom edges of the marker surfaces, the edges of the marker surfaces being proximal the upper and lower surfaces of the marker, respectively, and distal the centroid,
wherein the medial portions of the marker surfaces are further from each other than are the top and bottom edges of the marker surfaces, and
a tongue portion, comprising:
medial portions of sidewall surfaces extending parallel to the longitudinal axis, the medial portions of sidewall surfaces being located distal top and bottom surfaces of the element and proximal the centroid, and
top and bottom edges of the sidewall surfaces, the edges being proximal the upper and lower surfaces of the sidewalls, respectively, and distal the centroid,
wherein the medial portions of the sidewalls are further from each other than are the top and bottom edges of the sidewall surfaces.

17. The medical device of claim 16, wherein the medial portions of the marker surfaces are about 5%, about 3%, about 10%, 5-30% or 10-20% further from each other than are the top and bottom edges of the marker surfaces.

18. The medical device of claim 11, wherein a top and bottom surface of the marker each have a recessed area.

19. The medical device of claim 11, wherein a length of the marker extending parallel to the longitudinal axis of the element is greater than a width thereof measured perpendicular to the longitudinal axis.

20. The medical device of claim 11, wherein a top and bottom surface of the marker protrudes from a top and bottom opening, respectively of the hole by about 0%, about 5% or about 10%.
